(19) United States
(12) Patent Application Publication
(10) Pub. No.: US 2022/0152028 A1
(43) Pub. Date: May 19, 2022

(54) COMBINATION DRUG TREATMENT FOR HUMAN CYTOMEGALOVIRUS

(71) Applicant: Microbiotix, Inc., Worcester, MA (US)

(72) Inventors: Islam Hussein, Marlboro, MA (US); Jennifer L. Brooks, Kingston, NH (US); Terry L. Bowlin, Maineville, OH (US)

(21) Appl. No.: 17/438,452

(22) PCT Filed: Mar. 12, 2020
(86) PCT No.: PCT/US2020/022332

§ 371 Date: Sep. 12, 2021

Related U.S. Application Data
(60) Provisional application No. 62/817,069, filed on Mar. 12, 2019.

(51) Int. Cl. A61K 31/517; A61K 31/522; A61P 31/22

(52) U.S. Cl. CPC A61K 31/517; A61P 31/22; A61K 31/522

(57) ABSTRACT

The present invention is related to the discovery of a combination therapy for the treatment or prevention of cytomegalovirus infection in humans and other mammals, comprising the administration of a synergistic therapeutic effective amount of both filociclovir and letermovir.

### FIELD OF THE INVENTION

This invention is in the field of combination drug treatment of human cytomegalovirus. In particular, the present invention is directed to the discovery that a combination of the drugs filociclovir and letermovir has a synergistic effect when administered for the treatment or prevention of subjects infected with, or at risk of being infected with human cytomegalovirus.

### BACKGROUND OF THE INVENTION

Infection with human cytomegalovirus (HCMV), a β-herpesvirus, is very common in humans, with seroprevalence ranging between 50-90% depending on geography and socioeconomic status (Staras et al., Clin. Infect. Dis., 43(9): 1143-1151 (2006); Cannon et al., Rev. Med. Virol., 20(4): 202-213 (2010); Bate et al., Clin. Infect. Dis., 50(11): 1439-1447 (2010)). The virus establishes persistent infection, and patients typically remain asymptomatic. However, in immunocompromised patients, HCMV infection causes significant morbidity and mortality (Kovacs et al., N Engl. J. Med., 341(2): 77-84 (1999); Griffiths et al., J. Antimicrob. Chemother., 45 Suppl. T3: 29-34 (2000)). Up to 75% of solid organ transplant recipients develop CMV disease from either new infections or reactivation of latent virus after transplantation (Azevedo et al., Clinics, 70(7): 515-523 (2015)). Furthermore, HCMV is the most common congenitally-acquired infection worldwide (Boppana et al., Pediatrics, 104(1 Pt. 1): 55-60 (1999); Barbi et al., Pediatr. Infect. Dis. 22(1): 39-42 (2003); Demmler, G. J., Reviews of Infectious Diseases, 13(2): 315-329 (1991)). HCMV is the leading infectious cause of hearing loss, cognitive impairment, and CNS damage in children (Fowler, K B., Clin. Infect. Dis., 57 Suppl 4: S182-4 (2013); Kawasaki et al., Pathol. Int, 67(2): 72-82 (2017)).

Anti-HCMV drugs approved for therapy target the viral DNA polymerase. Ganciclovir (GCV), and its oral prodrug valganciclovir (VGCV), are first line drugs for treatment and prophylaxis. Secondary therapies include cidofovir (CDV) and foscarnet (FOS). Problems with dose-limiting toxicity, oral-bioavailability and resistance limit the usefulness and efficacy of currently-approved anti-HCMV therapies (Britt, W. J. and Prichard, M. N., Antiviral Res., 159: 153-174 (2018)). Recently, letermovir (LMV) was approved by the FDA for HCMV prophylaxis in stem cell transplant recipients (Marty et al., N. Engl. J. Med., 377: 2433-2444 (2017)). LMV targets the viral terminase complex, preventing the cleavage of concatemeric viral DNA into multiple unit length genomes (Griffiths, P. D. and Emery, V. C., New Engl. J. Med., 370(19): 1844-1846 (2014); Goldner et al., J. Virol., 85(20): 10884-10893 (2011)).

Maribavir (MBV) is another HCMV inhibitor that failed to meet clinical endpoints in a phase 3 prophylaxis trial (Marty et al., Lancet Infect. Dis., 11(4): 284-292 (2011)) but appeared active in a phase 2 treatment trial for refractory and resistant disease (Papanicolaou et al., Clin. Infect. Dis., 68: 1255-1264 (2019)).

Filociclovir (FCV) is a methylenecyclopropane nucleoside analogue, which has successfully completed human Phase I safety studies (Rouphael et al., Antimicrob. Agents Chemother., 63(9): e00717-19 (2019)) and is now entering Phase II human clinical efficacy studies for the treatment of HCMV-related disease in immunocompromised (e.g. transplant) patients. In HCMV-infected cells, FCV is phosphorylated by the virus-encoded UL97 kinase (Gentry et al., Antimicrob. Agents Chemother., 54(8): 3093-3098 (2010)). FCV-monophosphate (FCV-MP) is then converted to FCV triphosphate (FCV-TP) by cellular kinases (Li et al., Nucleosides, Nucleotides Nucleic Acids, 28(9): 795-808 (2009); Gentry et al., Biochem. Pharmacol., 81(1): 43-49 (2011)). FCV-TP reaches higher peak levels than GCV-TP in infected cells (Gentry, B. G. and Drach, J. C., Antimicrob. Agents Chemother., 58(4): 2329-2333 (2014)), and exhibits about 10-fold higher affinity to HCMV DNA polymerase UL54 (Chen et al., Antimicrob. Agents Chemother., 60(7): 4176-4182 (2016)). Although some GCV-resistant UL97 mutants of HCMV are cross-resistant to FCV, exonuclease and region V mutations at UL54 codons commonly associated with GCV-CDV cross-resistance in clinical isolates conferred increased susceptibility to FCV (Chou et al., Antimicrob. Agents Chemother., 56(1): 197-201 (2012)). Therefore, FCV may be a useful alternative therapy for treating patients infected with selected GCV-resistant HCMV strains.

Monotherapy may eventually select for drug-resistant strains, particularly in immunocompromised hosts, hence the need for combination therapies for chronic viral infections such as HIV and hepatitis (Shinazi et al., Liver Int, 34 Suppl. 1: 69-78 (2014); DeClercq, E., Nat. Rev. Drug Discov., 6: 1001-1018 (2007)). Although LMV is highly potent with EC₅₀ values in the nanomolar range, high-grade resistance mutations in the UL56 terminase gene were shown to readily emerge in vitro (Goldner et al., Antimicrob. Agents Chemother., 58(1): 610-613 (2014)) and in treated patients (Marty et al., N. Engl. J. Med., 377: 2433-2444 (2017); Lischka et al., J. Infect. Dis., 213: 23-30 (2016)), raising concerns about LMV monotherapy. Unlike LMV, which is highly specific for HCMV (Marschall et al., Antimicrob. Agents Chemother., 56(2): 1135-1137 (2012)), FCV has a broader spectrum and is active against several viruses posing significant risk to transplant patients (Hartline et al., Antiviral Res., 159: 104-112 (2018); Prichard et al., Antimicrob. Agents Chemother., 57(8): 3518-3527 (2013)). Also, the favorable safety profile of both drugs and the different mechanisms of action greatly reduce the potential for cross-resistance. This makes FCV a particularly attractive candidate for LMV drug combinations prescribed for both prevention and treatment indications.

Therefore, currently there is a need for a safe and effective method for the treatment and/or prevention of HCMV infection. A composition comprising a synergistic combination of LMV and FCV is of particular interest, given the favorable safety profile of both drugs and the different mechanisms of action. As set forth below, we demonstrate an unexpected synergistic effect between FCV and LMV in vitro and show that FCV is highly potent against HCMV strains carrying 4 mutations at 3 UL56 codons that are commonly associated with LMV resistance (Marty et al., supra; Goldner et al., supra; Chou et al., supra; Lischka et al., supra).

### SUMMARY OF THE INVENTION

The present invention is directed to a novel method for treating a viral disease by administration of a therapeutic combination of drugs to provide a safe, effective, and potent treatment that is more effective than administration of either drug alone and demonstrates an improved therapeutic synergistic effect beyond what would be expected from administering the drug combination. In particular, the present invention is directed to the discovery of a novel method for treating or preventing cytomegalovirus infection, and in particular, human cytomegalovirus (HCMV) infections. The novel method comprising administering a synergistic therapeutic effective amount of a combination of filociclovir (FCV) and letermovir (LTV) to a subject in need thereof. We demonstrate herein that administration of a combination of both filociclovir and letermovir is effective to treat or prevent HCMV infections that is more potent than what would be expected from administration of either of these drugs individually. In other words, the method for the combined administration of filociclovir and letermovir as described herein is more effective than the theoretical combined additive effect observed when the inhibitory properties, e.g., EC₅₀ value, of each compound is tested separately or individually against cytomegalovirus. In other words, the novel method described herein for the treatment or prevention of HCMV infection by administration of the filociclovir/letermovir drug combination demonstrates an unexpected level of effectiveness beyond what would be expected by combining these two compounds, i.e., the drug combination exhibits a “synergistic” therapeutic effect. The novel method described herein is suitable for the treatment and/or prevention of cytomegalovirus infections in mammals, and in particular for treating or preventing cytomegalovirus infections in humans.

In another embodiment, the present invention is directed to the use of filociclovir and letermovir in the manufacture of a medicament comprising a therapeutic combination of filociclovir and letermovir for use in a method for treating or preventing cytomegalovirus, in particular HCVM infection in a mammal. In a preferred embodiment, the mammal is a human.

Filociclovir and letermovir may be formulated in the same composition or formulated in separate compositions for later combination or alternatively formulated in separate compositions that may be administered concurrently, simultaneously, serially, and/or separately at timed intervals. Multiple doses of the formulation or formulations are also contemplated.

Also disclosed are pharmaceutical compositions comprising a synergistic therapeutically effective amount of filociclovir, or a pharmaceutically acceptable salt thereof, and a synergistic therapeutically effective amount of letermovir, or a pharmaceutically acceptable salt thereof, and a pharmaceutically acceptable carrier. The pharmaceutical compositions may comprise a combination of filociclovir and letermovir for simultaneous administration of both compounds. Alternatively, filociclovir and letermovir may be formulated in separate pharmaceutical compositions in which case the synergistic therapeutic drug combination may be administered in concurrent or simultaneous doses or serially dosed at timed intervals. For example, for serial administration of the drug combination, the pharmaceutical composition comprising filociclovir may be administered before the pharmaceutical composition comprising letermovir and the pharmaceutical composition comprising letermovir is then administered within a certain time period thereafter, or vice versa where the pharmaceutical composition comprising letermovir is administered before the pharmaceutical composition comprising filociclovir, in either case the improved therapeutic benefit of administering the drug combination is maintained.

The pharmaceutical compositions are suitable for use in the disclosed methods for treating or preventing HCMV infections in a mammal, particularly in humans. The pharmaceutical compositions may be formulated for both parenteral and/or nonparenteral administration to a subject or patient in need thereof.

In another embodiment, the drug combination comprising filociclovir and letermovir described herein may be administered to a subject in need thereof optionally in combination with one or more additional antiviral agents. The additional antiviral agent or agents may be administered before, simultaneously with, or after administration of the filociclovir/letermovir synergistic therapeutic drug combination described herein.

### Definitions

As used herein, the term “synergistic” or “synergistic effect” or “synergistic therapeutic effect” or “synergistic therapeutic effective amount” or “synergistic prophylactic effective amount”, and variations thereof, refers to the concentration of each antiviral compound that, when combined in a pharmaceutical formulation and administered to a subject, or formulated in separate pharmaceutical formulations and administered in combination to a subject, either at the same time, simultaneously, concurrently, or serially, has a beneficial effect in treating, preventing, or otherwise ameliorating a viral infection in a subject without being antagonistic or toxic to the subject and shows an improved effect over what is observed or would be expected by the sum of their individual effects from administration of either antiviral compound alone (synergistic effect).

A composition or method described herein as “comprising” (or “comprises”) one or more named elements or steps is open-ended, meaning that the named elements or steps are essential, but other elements or steps may be added within the scope of the composition or method. To avoid prolixity, it is also understood that any composition or method described as “comprising” one or more named elements or steps also describes the corresponding, more limited, composition or method “consisting essentially of” (or “consists essentially of”) the same named elements or steps, meaning that the composition or method includes the named essential elements and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other element or step. In any composition or method disclosed herein, known or disclosed equivalents of any named essential element or step may be substituted for that element or step, respectively.

As used herein, the term “subject” can be a human, non-human primate, horse, pig, rabbit, dog, sheep, goat, cow, cat, guinea pig or rodent. A “patient” or “subject in need thereof” refers to a mammal afflicted with a disease or disorder. The term “patient” includes human and veterinary subjects.

Terms such as “parenteral”, “parenterally”, and the like, refer to routes or modes of administration of a compound or composition to an individual other than along the alimentary canal. Examples of parenteral routes of administration include, without limitation, subcutaneous (s.c.), intravenous (i.v.), intramuscular (i.m.), intra-arterial (i.a.), intraperitoneal (i.p.), transdermal (absorption through the skin or dermal layers), nasal (‘intranasal’; absorption across nasal mucosa), or pulmonary (e.g., inhalation for absorption across the lung tissue), vaginal, direct injections or infusions into body cavities or organs other than those of the alimentary canal, as well as by implantation of any of a variety of devices into the body (e.g., of a composition, depot, or device that permits active or passive release of a compound or composition into the body).

The terms “non-parenteral”, “non-parenterally”, “enteral”, “enterally”, “oral”, “orally”, and the like, refer to administration of a compound or composition to an individual by a route or mode along the alimentary canal. Examples of enteral routes of administration include, without limitation, oral, as in swallowing solid (e.g., tablet) or liquid (e.g., syrup) dosage forms, sublingual (absorption through the mucosal membranes lining the floor of the mouth, e.g., under the tongue), buccal (absorption through the mucosal membranes lining the cheeks), nasojejunal or gastrostomy tubes (delivery into the stomach), intraduodenal administration, as well as rectal administration (e.g., suppositories for release of a drug composition into and absorption by the lower intestinal tract of the alimentary canal).

### BRIEF DESCRIPTION OF THE DRAWINGS

FIGS. 1(a) and (b) show the results of the inhibitory assay testing the effects of various concentrations of filociclovir and letermovir against human cytomegalovirus (HCMV) strain AD169. FIG. 1(a) shows the dose-response curves for filociclovir (top) and letermovir (bottom) tested separately in HFF cells infected with HCMV. FIG. 1(b) is a 3D Synergy plot showing the inhibitory effect of filociclovir and letermovir administered as a drug combination in HFF cells infected with HCMV. The two peaks shown above 0% represent the concentrations at which the filociclovir and letermovir drug combination acts synergistically to inhibit HCMV infection. The single peak below 0% represents the concentration at which the filociclovir and letermovir drug combination acts antagonistically.

### DETAILED DESCRIPTION OF THE INVENTION

Infection with human cytomegalovirus (HCMV), a β-herpesvirus, is very common in humans, with seroprevalence ranging between 50-90% depending on geography and socioeconomic status (Staras et al., Clin.l Infect. Dis., 43(9): 1143-1151 (2006); Cannon et al., Rev. Med. Virol. 20(4): 202-213 (2010); Bate et al., Clin. Infect. Dis., 50(11): 1439-1447 (2010)). The virus establishes persistent infection, and patients typically remain asymptomatic. However, in immunocompromised patients, HCMV infection causes significant morbidity and mortality (Kovacs et al., N. Engl. J. Med., 341(2): 77-84 (1999); Griffiths et al., J. Antimicrob. Chemother., 45 Suppl T3: 29-34 (2000)). Up to 75% of solid organ transplant recipients develop CMV disease from either new infections or reactivation of latent virus after transplantation (Azevedo et al., Clinics, 70(7): 515-523 (2015)). Furthermore, HCMV is the most common congenitally-acquired infection worldwide (Boppana et al., Pediatrics, 104(1 Pt 1): 55-60 (1999); Barbi et al., Pediatr. Infect. Dis. 22(1): 39-42 (2003); Demmler, G. J., Rev. Infect. Dis., 13(2): 315-329 (1991)). HCMV is the leading infectious cause of hearing loss, cognitive impairment, and CNS damage in children (Fowler, K. B., Clin. Infect. Dis., 57 Suppl 4: S182-4 (2013); Kawasaki et al., Pathol. Int., 67(2): 72-82 (2017)). Therefore, currently there is a need for a safe and effective method for treating HCMV.

Herein we describe the discovery of a novel method for treating and/or preventing viral infection in a mammal by administration of a therapeutic combination of filociclovir (FCV) or a pharmaceutically acceptable salt thereof and letermovir (LMV) or a pharmaceutically acceptable salt thereof. In a preferred embodiment, the mammal is a human.

Filociclovir, ((Z)-9-{[2,2-bis-(hydroxymethyl)cyclopropylidene]methyl}guanine), is a methylenecyclopropane nucleoside analog having the following structure:

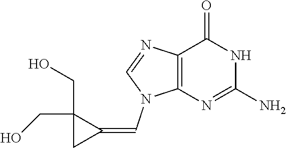 [SMILES: Nc1nc2c(ncn2/C=C2/CC2(CO)CO)c(=O)[nH]1]

Letermovir, 2-[(4S)-8-fluoro-2-[4-(3-methoxyphenyl)piperazin-1-yl]-3-[2-methoxy-5-(trifluoromethyl)phenyl]-4H-quinazolin-4-yl]acetic acid, is an antiviral agent that inhibits HCMV replication by binding to components of the terminase complex, UL51, UL56, or both. Letermovir has the following structure:

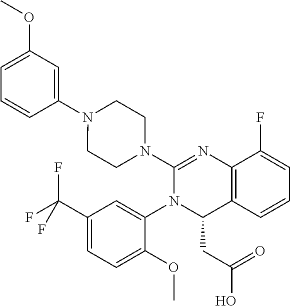 [SMILES: COc1cccc(N2CCN(C3=Nc4c(F)cccc4[C@H](CC(=O)O)N3c3cc(C(F)(F)F)ccc3OC)CC2)c1]

The results described herein demonstrate that administration of a combination of filociclovir and letermovir is more effective for treating and or preventing cytomegalovirus infection, in particular human cytomegalovirus (HCMV) infection than administration of either compound alone or individually and shows an improved (synergistic) therapeutic effect at inhibiting HCMV over what would be expected from administration of the combination based on the observed sum or expected additive effect of the viral inhibitory properties of each compound administered separately or individually. In other words, the results seen by administering the filociclovir/letermovir drug combination as disclosed herein is not merely “additive” of the inhibitory properties, e.g., EC₅₀ values, observed for administering each compound individually, but rather the effectiveness at treating and/or preventing HCMV by administering a combination of these two drugs exceeds the level of the expected additive effect and unexpectedly demonstrates a beneficial synergistic effect above and beyond the calculated theoretical additive effect.

In one embodiment, the pharmaceutical composition according to the present invention comprises filociclovir and letermovir formulated in the same composition, thus allowing for simultaneous administration of both compounds in a single dose. Multiple doses of this composition are also contemplated.

Alternatively, filociclovir and letermovir may be formulated in separate pharmaceutical compositions, wherein the first pharmaceutical composition comprises filociclovir and the second pharmaceutical composition comprises letermovir, or vice versa. According to this embodiment, the separate formulations may be administered simultaneously, or they may be administered sequentially, or serially in either order at various time intervals. For example, if the pharmaceutical formulation comprising filociclovir is administered first, the pharmaceutical formulation comprising letermovir may be administered sequentially or serially thereafter, for example, within 24 hours, less than 12 hours, less than 8 hours, less than 6 hours, less than 4 hours, less than 2 hours, less than 1 hour, less than 30 minutes, less than 10 minutes, less than 1 minute, or immediately after administration of the pharmaceutical formulation comprising filociclovir, or vice versa if the pharmaceutical formulation comprising letermovir is administered before the pharmaceutical formulation comprising filociclovir. Multiple doses of these formulations simultaneously, sequentially, or serially in either order are also contemplated.

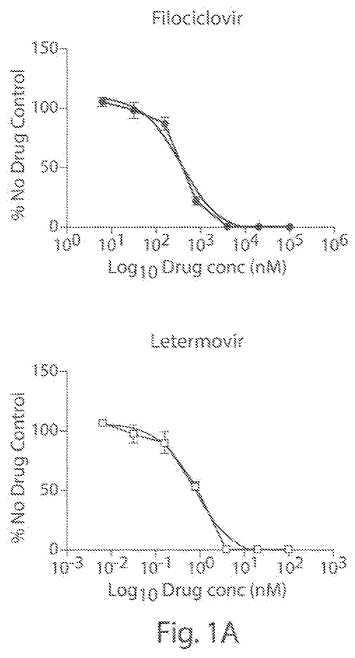

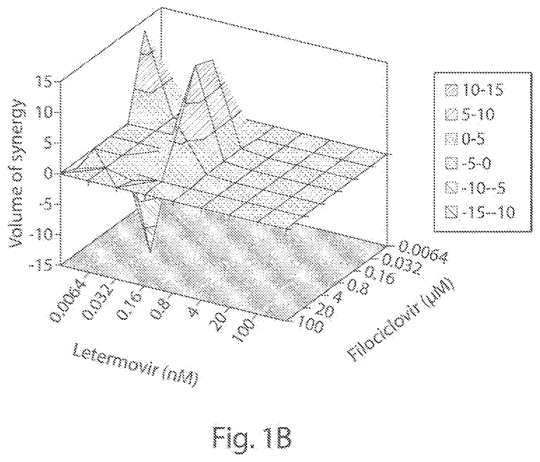

In a preferred embodiment, the method of the present invention is effective to treat or prevent infection by multiple strains of cytomegalovirus. As described below, using a standard assay to measure the inhibitory effects of filociclovir and letermovir against HCMV-infected mammalian cells, it has been demonstrated that administration of a combination of filociclovir and letermovir exhibits an unexpectedly improved therapeutic effect against human cytomegalovirus (HCMV) strain AD169, than administration of either compound alone as seen in the 3D synergy plot shown in FIG. 1(b). FIG. 1(a) shows the inhibitory effect of each compound tested separately, filociclovir (top graph) and letermovir (bottom graph), in mammalian cells infected with HCMV strain AD169. The results shown in FIGS. 1(a) and 1(b) demonstrate that administration of filociclovir and letermovir as a drug treatment combination (FIG. 1(b)) against cytomegalovirus as described herein is more effective than what would be expected by the theoretical “additive” effect based on the results (EC₅₀) observed from individual administration of each compound as shown in FIG. 1(a).

The present invention is further directed to the use of a formulation comprising the filociclovir and letermovir drug combination to treat or prevent viral cytomegalovirus infection, particularly human cytomegalovirus infections. The present invention further relates to the use of filociclovir, or a pharmaceutically acceptable salt thereof, and letermovir, or a pharmaceutically acceptable salt thereof, in the manufacture of a medicament for use in a method for treating or preventing cytomegalovirus infection in mammals (e.g., humans), the method comprising the combined administration of said filociclovir or pharmaceutically acceptable salt thereof and said letermovir or pharmaceutically acceptable salt thereof, with a pharmaceutically acceptable carrier or diluent.

In another aspect, the invention relates to a kit comprising at least filociclovir, or a pharmaceutically acceptable salt, solvate, or polymorph thereof, and letermovir, or a pharmaceutically acceptable salt, solvate, or polymorph thereof. In one embodiment, the pharmaceutical formulation comprising filociclovir and the pharmaceutical formulation comprising letermovir may be in the same vial or container, or alternatively, may be in separate vials or containers. The kit includes one or more of:
a) optionally at least one additional agent known to have antiviral activity;
b) instructions for treating a cytomegalovirus related disease;
c) instructions for formulating, combining, and/or administering the composition comprising filociclovir and the composition comprising letermovir in connection with treating a cytomegalovirus infection; or
d) instructions for administering the compound with at least one agent known to treat a cytomegalovirus related disease.

In a further aspect, the kit further comprises a plurality of dosage forms of filociclovir, or a pharmaceutically acceptable salt, solvate, or polymorph thereof, and letermovir, or a pharmaceutically acceptable salt, solvate, or polymorph thereof, the plurality comprising one or more doses of each compound; wherein each dose comprises a therapeutically effective amount of filociclovir and a therapeutically effective amount of letermovir suitable for the synergistic combination of each according to the present invention. It is also contemplated that the drug combination of filociclovir and letermovir may be formulated as separate doses of each compound and administered simultaneously or serially (e.g., either administration of the formulation comprising filociclovir first followed by administration of the formulation comprising letermovir, or vice versa).

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound of the present invention and/or product and another component for delivery to a patient.

In a further aspect, an effective amount of the combination of filociclovir and letermovir according to the present invention is a synergistic therapeutically effective amount. In a still further aspect, an effective amount of the combination of filociclovir and letermovir according to the present invention is a synergistic prophylactically effective amount.

The drug combination of the present invention may comprise filociclovir at a concentration of from about 1 nM to about 1 mM and letermovir at a concentration of from about 0.0001 nM to about 100 nM formulated in a pharmaceutically acceptable carrier or excipient.

Either or both of filociclovir and letermovir can be administered as a pharmaceutically acceptable salt. Such pharmaceutically acceptable salts include the gluconate, lactate, acetate, tartarate, citrate, phosphate, maleate, borate, nitrate, sulfate, and hydrochloride salts. The salts of the compounds described herein can be prepared, for example, by reacting the base compound with the desired acid in solution. After the reaction is complete, the salts are crystallized from solution by the addition of an appropriate amount of solvent in which the salt is insoluble. In some embodiments, the hydrochloride salt is made by passing hydrogen chloride gas into an ethanolic solution of the free base. Accordingly, in some embodiments, the pharmaceutically acceptable salt is a hydrochloride salt.

In another embodiment, the compounds are formulated into a pharmaceutically acceptable carrier or excipient for administration to a subject in need thereof. In another embodiment, the compounds may be formulated into a pharmaceutical formulation and further comprise an additional antiviral compound. In another embodiment, the pharmaceutical formulation may be formulated to be administered orally, parenterally, or topically.

### Compositions and Methods

Pharmaceutical compositions or formulations according to the present invention comprise the compounds, filociclovir and letermovir, as described herein, or a pharmaceutically acceptable salt, solvate, or polymorph thereof, as the ‘active ingredient’ and a pharmaceutically acceptable carrier (or ‘vehicle’), which may be a liquid, solid, or semi-solid compound.

It is preferable to develop an orally active therapeutic, since that is the most convenient and rapid method to administer a drug to an exposed individual, individuals, or population. However, it is also expected that the drug combination described herein will be suitable for intravenous (i.v.) administration. Therefore, the pharmaceutical formulations described herein will provide an effective, safe, and easy therapeutic option for treating or preventing cytomegalovirus infection.

As used herein, the terms “treat”, “treating”, and grammatical variations thereof, as well as the phrase “method of treating” and “method of preventing”, are meant to encompass any desired therapeutic intervention, including but not limited to a method for treating an existing cytomegalovirus infection in a subject, and a method for the prophylaxis (i.e., preventing) of cytomegalovirus infection, such as in a subject that has been exposed to cytomegalovirus as disclosed herein or that has an expectation of being exposed to the cytomegalovirus as disclosed herein.

In another embodiment, filociclovir and letermovir may be formulated into a pharmaceutically-acceptable carrier and applied/administered to a subject in need thereof by an injection, including, without limitation, intradermal, transdermal, intramuscular, intraperitoneal and intravenous.

According to another embodiment of the invention, the administration is oral and the composition may be presented, for example, in the form of a tablet or encased in a gelatin capsule or a microcapsule, which simplifies oral application. The production of these forms of administration is within the general knowledge of a technical expert. Multiple routes of administration are envisioned for these drug-like molecules, and highly cost-effective production strategies can be easily achieved.

In a further aspect, the pharmaceutical formulation of the present invention is a solid dosage form selected from a capsule, a tablet, a pill, a powder, a granule, an effervescing granule, a gel, a paste, a troche, and a pastille. In a still further aspect, the pharmaceutical formulation is a liquid dosage form selected from an emulsion, a solution, a suspension, a syrup, and an elixir.

As used herein, the term “pharmaceutically acceptable salts” refers to salts prepared from pharmaceutically acceptable non-toxic bases or acids. When the composition of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In preparing the formulations for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical formulations of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods.

Pharmaceutical formulations of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions of the invention, and/or pharmaceutically acceptable salts thereof, can also be prepared in powder or liquid concentrate form.

The pharmaceutical formulations of the present invention may comprise a therapeutically effective amount of filociclovir and letermovir combined together into a single formulation for administration or they may be formulated in separate formulations, wherein the first formulation comprises filociclovir and the second formulation comprises letermovir. According to this embodiment, the separate formulations may be administered at the same time, concurrently, simultaneously, or serially one after the other in any order or timed interval, i.e., the formulation comprising filociclovir may be administered before the formulation comprising letermovir, or vice versa, at timed intervals so long as the combination of filociclovir and letermovir exhibits a therapeutic effect according to the present invention to treat or prevent cytomegalovirus infection in a subject, for example, a human subject. For example, if the pharmaceutical formulation comprising filociclovir is administered first, the pharmaceutical formulation comprising letermovir may be administered separately thereafter, for example, within 24 hours, less than 12 hours, less than 8 hours, less than 6 hours, less than 4 hours, less than 2 hours, less than 1 hour, less than 30 minutes, less than 10 minutes, less than 1 minute, or immediately after administration of the pharmaceutical formulation comprising filociclovir, or vice versa. Multiple doses of these formulations whether formulated separately or together are also contemplated.

In another aspect, the present invention relates to a kit comprising at least filociclovir and letermovir according to the present invention, or a pharmaceutically acceptable salt, solvate, or polymorph thereof; and one or more of:
a) optionally at least one additional agent known to have antiviral activity;
b) instructions for treating a cytomegalovirus-related disease;
c) instructions for formulating and/or administering the composition in connection with treating a cytomegalovirus infection; or
d) instructions for administering the compound with at least one agent known to treat a cytomegalovirus related disease.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound or compounds of the present invention and/or product and another component for delivery to a patient.

In a further aspect, the kit further comprises a plurality of dosage forms, the plurality comprising one or more doses; wherein each dose comprises an amount of the compound or compounds.

In a further aspect, an effective amount is a therapeutically effective amount. In a still further aspect, an effective amount is a prophylactically effective amount.

### Examples

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1. Assay to Measure Inhibition of Cytomegalovirus by Filociclovir and Letermovir and Determination of the Synergistic Concentration of a Composition Comprising Both Compounds

We examined the in vitro anti-HCMV (strain AD169; ATCC #VR-538) activity of filociclovir and letermovir both separately and as triplicate combinations at a concentration range of 0.0064-100 μM, and 0.0064-100 nM, respectively.

Monolayers of human foreskin fibroblasts (HFF cells; ATCC #SCRC-1041) were grown in 1 ml of DMEM (Corning #10-013-CM) containing 10% heat-inactivated fetal bovine serum (Gibco #A38402-01) and 1% penicillin-streptomycin solution (Hyclone #SV30010) in 24-well plates and infected with 100 plaque forming units (PFUs) of HCMV. Following incubation for 1 hr at 37° C., the virus was aspirated and replaced with MEM (Corning #50-010-PB) containing 1% methyl cellulose (Sigma #M-0521) and the various concentrations of filociclovir and letermovir were added as set forth above. A set of wells, designated as non-treated controls, received neither of the two compounds.

Infected plates were incubated for 9 days at 37° C., then fixed and stained with crystal violet stain (0.1% in 20% methanol). The number of plaques in each well was counted and the level of synergism was assessed using MacSynergy™ II (Prichard and Shipman 1990, supra), which provides an overall evaluation of the deviation from the theoretical additive effect of both drugs based on individual dose-response curves.

### Results

As shown in FIG. 1(a), the individual calculated EC₅₀ values for filociclovir and letermovir tested separately were 550 nM and 3 nM, respectively, which is consistent with previous values reported in the literature (Hartline et al., Antiviral Res., 159: 104-112 (2018); Chou S., Antiviral Res., 148: 1-4 (2017)).

Analysis of the inhibitory properties of the filociclovir/letermovir tested in combination at various concentrations demonstrated a synergistic effect using MacSynergy™ II which calculated the volume of synergy at 57 at a confidence interval of 95% (FIG. 1(b)), which is a statistically significant value. Synergy values between 50 and 100 indicate moderate synergy. Therefore, the combination of filociclovir and letermovir offers a promising treatment or prevention option against cytomegalovirus, e.g., human cytomegalovirus infection.

As seen in FIG. 1(b), the two peaks above the plane in the range 5-15 represent the concentrations at which the combination of filociclovir and letermovir act synergistically. The single peak below the plane represents the concentrations at which the combination of filociclovir and letermovir are antagonistic.

### CONCLUSIONS

Therefore, the 3D synergy plot shown in FIG. 1(b) demonstrates that administration of filociclovir and letermovir as a drug combination against cytomegalovirus infection unexpectedly shows a beneficial synergistic effect that is an improvement over the theoretical additive effect when the inhibitory properties of each compound is tested separately as evidenced by the data shown in FIG. 1(a).

### Example 2. Assay to Measure Effect of Filociclovir on Letermovir-Resistant Strains

In order to test the effect of filociclovir on letermovir-resistant strains, we used 4 HCMV UL56 mutants encountered in clinical practice: V231L, V236M, C325F and C325Y, the latter 2 of which commonly emerged to confer absolute letermovir resistance (Grantham et al., Biol. Blood Marrow Transplant. 25: S344-S345 (2019); Turner et al., Antimicrob. Agents Chemother., 63(3): e02337-18 (2019)).

These UL56 amino acid substitutions were introduced into the bacterial artificial chromosome (BAC) clone BD2 of HCMV laboratory strain AD169 modified with a secreted alkaline phosphatase (SEAP) reporter gene, as previously described (Chou, S., Antimicrob. Agents Chemother., 59: 6588-6593 (2015)). The mutagenized BAC clones were transfected into modified retinal epithelial (ARPEp) cells and the recovered infectious recombinant viruses were then genotyped and phenotyped as previously described (Chou et al., Antimicrob. Agents Chemother., 62(9): e00922-18 (2018)).

Control strains included a baseline strain of wild-type UL56 and a UL97 M460V mutant that is resistant to filociclovir. For susceptibility assays, calibrated viral inocula were added to 24-well plate cultures of ARPEp cells and incubated under a series of filociclovir concentrations, and the concentrations required to reduce SEAP activity by 50% (EC₅₀) at 6 days were determined in duplicate assays on each of 3 to 5 setup dates. The results are shown in Table 1.

As seen in Table 1, filociclovir was more potent against the UL56 mutants tested than against wild type virus, with mean EC₅₀ values ranging from 0.29-0.38 μM (wildtype EC₅₀=0.43 μM). This highlights a promising therapeutic benefit for including filociclovir in letermovir regimens.

TABLE 1
Susceptibility of HCMV control strains
and UL56 mutants to Filociclovir

| | | Mean | Standard | Number of | |
|---|---|---|---|---|---|
| | Genotype | EC₅₀ (μM) | Deviation | replicates | Ratio* |
| Control | UL56 wt | 0.43 | 0.04 | 10 | — |
| strains | UL97 M460V | 2.03 | 0.23 | 10 | 4.8 |
| UL56 | V231L | 0.29 | 0.06 | 6 | 0.69 |
| mutants | V236M | 0.35 | 0.06 | 8 | 0.81 |
| | C325F | 0.35 | 0.07 | 6 | 0.82 |
| | C325Y | 0.38 | 0.06 | 8 | 0.89 |

*Ratio = EC₅₀ of mutant/EC₅₀ of wild type control

Consideration of the foregoing data shows that the combination of filociclovir and letermovir is an effective antiviral agent, leading to the conclusion that this drug combination is more effective than administration of either compound alone in treating or preventing viral infection and shows an unexpectedly improved synergistic therapeutic effect for treating or preventing viral infection.

All publications, patent applications, patents, and other documents cited herein are incorporated by reference in their entirety. The example set forth above is illustrative only and not intended to be limiting. Obvious variations to the disclosed methods and alternative embodiments of the invention will be apparent to those skilled in the art in view of the foregoing disclosure. All such obvious variants and alternatives are considered to be within the scope of the invention as described herein.

What is claimed is:

1. A method for treating or preventing a virus infection in a mammal comprising administering to said mammal a synergistic therapeutic effective amount of filociclovir or a pharmaceutically acceptable salt thereof and letermovir or a pharmaceutically acceptable salt thereof.

2. The method according to claim 1, wherein said virus is human cytomegalovirus.

3. The method according to claim 1, wherein said filociclovir and said letermovir are administered simultaneously.

4. The method according to claim 1, wherein said filociclovir and said letermovir are administered sequentially or serially.

5. The method according to claim 1, wherein the mammal is a human.

6. Use of a synergistic therapeutic effective amount of filociclovir and letermovir in the manufacture of a medicament for use in treating or preventing a virus infection in a mammal.

7. The use according to claim 6, wherein said virus is cytomegalovirus.

8. The use according to claim 6, wherein said mammal is a human.

9. A pharmaceutical composition, or pharmaceutically acceptable salt thereof, comprising a synergistic therapeutic effective amount of filociclovir and letermovir for treating or preventing a cytomegalovirus infection.

10. The pharmaceutical composition according to claim 9, wherein said cytomegalovirus is human cytomegalovirus.

11. Use of a synergistic therapeutic effective amount of filociclovir and letermovir in combination for treating or preventing cytomegalovirus infection.

12. The use according to claim 11, wherein said cytomegalovirus is human cytomegalovirus.
